(19) United States
(12) Patent Application Publication
(10) Pub. No.: US 2025/0066293 A1
(43) Pub. Date: Feb. 27, 2025

(54) OIL-BASED ISOTHIOCYANATE PRODUCTS AND PROCESSES FOR PREPARING OIL-BASED ISOTHIOCYANATE PRODUCTS

(71) Applicant: BRASSICA PROTECTION PRODUCTS LLC, Baltimore, MD (US)

(72) Inventors: Jed Fahey, Baltimore, MD (US); Ralf Schlothauer, Baltimore, MD (US)

(73) Assignee: BRASSICA PROTECTION PRODUCTS LLC, Baltimore, MD (US)

(21) Appl. No.: 18/727,010

(22) PCT Filed: Jan. 6, 2023
(86) PCT No.: PCT/US2023/060217

§ 371 Date: Jul. 5, 2024

Related U.S. Application Data
(60) Provisional application No. 63/297,010, filed on Jan. 6, 2022.

(51) Int. Cl. C07C 331/20; A23L 33/105

(52) U.S. Cl. CPC C07C 331/20; A23L 33/105

(57) ABSTRACT

Provided herein is an oil-based composition which contains high concentrations of isothiocyanates, which is shelf-stable at room temperature. Also provided herein is a method of extraction of isothiocyanates derived from the glucosinolates in plants that contain them, into an oil. The process described herein utilizes no organic solvents or solubilizing agents.

### BACKGROUND

Described herein is a process for extraction of isothiocyanates from glucosinolate-containing plants, and preparation of an oil-based composition comprising isothiocyanates. This process provides a product comprising isothiocyanates in oil which includes higher concentrations than are obtainable using prior art methods, without employing organic solvents or solubilizing agents.

Glucosinolates are highly water-soluble, stable compounds, which are converted to isothiocyanates by the enzyme myrosinase, which co-exists within the plant tissues. Isothiocyanates are less water soluble than glucosinolates and have a range of fat solubilities (lipophilicity). The myrosinase enzymatic reaction (conversion of glucosinolate precursors to isothiocyanates) occurs only in hydrated conditions, and this reaction produces a less water soluble, but biologically active compound from one that is highly water soluble, and relatively non-reactive.

This method confers stability to the isothiocyanate, thereby providing a new and effective vehicle for administering the natural, oil-based isothiocyanate formulation to living systems such as animals, microbes, fungi and plants. The combined stability and properties of the extracted isothiocyanate in oil expands the potential for different types of applications, including but not limited to, dermal absorption, inhalation (nasal sprays), ingestion, injection, and spray application. Further, the natural, oil-based delivery vehicle enhances the potential commercial use of the isothiocyanate oil extract either alone or as a component in the compositions of pharmaceutical products, food or drink products, dietary supplements and additives, skin or hair products, and agricultural products.

### SUMMARY

Described herein is a method of extracting glucosinolates from glucosinolate-containing plant material without the need for organic solvents or solubilizing agents. The glucosinolates may then be converted to isothiocyanates by exposure to the enzyme myrosinase; the resulting aqueous isothiocyanate-containing concentrate may be further extracted into a vegetable oil, mineral oil, or other oil, providing an oil which is rich in natural isothiocyanates.

The isothiocyanate may be one or more of 1-acetyl-indol-3-methyl isothiocyanate; 4-(4′-O-acetyl-α-L-rhamnosyloxy)benzyl isothiocyanate; 3-O-apiosylglucomatronalin isothiocyanate; 3-O-apiosylglucomatronalin 3,4-dimethosybenzoyl ester isothiocyanate; 2-(α-L-arabinopyranosyloxy)-2-phenylethyl isothiocyanate; 4-(benzoyloxy)butyl isothiocyanate; 2-(benzoyloxy)ethyl isothiocyanate; 6-(benzoyloxy)hexyl isothiocyanate; 5-(benzoyloxy)pentyl isothiocyanate; 2-benzolyloxy-1-ethylethyl isothiocyanate; benzoyloxymethyl isothiocyanate; 2-benzoyloxy-1-methylethyl isothiocyanate; 3-benzoyloxypropyl isothiocyanate; benzyl isothiocyanate; but-3-enyl isothiocyanate; n-butyl isothiocyanate; 3,4-dihydroxybenzyl isothiocyanate; 3,4-dimethoxybenzyl isothiocyanate; 2,2-dimethyl-2-(4-methoxyphenyl)ethyl or 2-(4-methoxyphenyl)-2,2-dimethylethyl isothiocyanate; ethyl isothiocyanate; 1-ethyl-2-hydroxyethyl isothiocyanate; 4-(α-L-glucopyranosyloxy)benzyl) isothiocyanate; 6-heptenyl isothiocyanate; 5-hexenyl isothiocyanate; n-hexyl isothiocyanate; 2-hydroxybenzyl isothiocyanate; 3-hydroxybenzyl isothiocyanate; 4-hydroxybenzyl isothiocyanate; 2(R)-hydroxybut-3-enyl isothiocyanate; 2(S)-hydroxybut-3-enyl isothiocyanate; 3-hydroxybutyl isothiocyanate; 4-hydroxybutyl isothiocyanate; 2-hydroxyethyl isothiocyanate; 4-hydroxyindol-3-ylmethyl isothiocyanate; 2-hydroxy-2-methylbutyl isothiocyanate; 1-(hydroxymethyl)propyl isothiocyanate; 2-hydroxy-2-methylpropyl isothiocyanate; 3-hydroxy-6-(methylsulfinyl)hexyl isothiocyanate; 3-hydroxy-5-(methylsulfinyl)pentyl isothiocyanate; 3-hydroxy-6-(methylsulfonyl)hexyl isothiocyanate; 3-hydroxy-5-(methylsulfonyl)pentyl isothiocyanate; 3-hydroxy-6-(methylthio)hexyl isothiocyanate; 3-hydroxy-5-(methylthio)pentyl isothiocyanate; 2-hydroxypent-4-enyl isothiocyanate; 2-hydroxypentyl isothiocyanate; 2(R)-hydroxy-2-phenylethyl isothiocyanate; 2-hydroxypropyl isothiocyanate; 3-hydroxypropyl isothiocyanate; indol-3-ylmethyl isothiocyanate; “iso”-heptyl isothiocyanate; “iso”-hexyl isothiocyanate; 2-methoxybenzyl isothiocyanate; 3-methoxybenzyl isothiocyanate; 4-methoxybenzyl isothiocyanate; 1-methoxyindol-3-ylmethyl isothiocyanate; 4-methoxyindol-3-ylmethyl isothiocyanate; 2-(4-methoxyphenyl)-2-hydroxyethyl isothiocyanate; methyl isothiocyanate; 3-methyl-3-butenyl isothiocyanate; 1-methylbutyl isothiocyanate; 2-methylbutyl isothiocyanate; 3-methylbutyl isothiocyanate; 3-methylcarbonylpropyl isothiocyanate; 1-methylethyl (isopropyl) isothiocyanate; 1-methyl-2-hydroxyethyl isothiocyanate; 3-methylpentyl isothiocyanate; 4-methylpentyl isothiocyanate; 2-(4-methoxyphenyl)-2-hydroxyethyl isothiocyanate; 2-methyl-2-propenyl isothiocyanate; 1-methylpropyl (sec-butyl, 2-butyl) isothiocyanate; 2-methylpropyl (isobutyl, 2-propyl) isothiocyanate; 4-methylsulfinylbut-3-enyl isothiocyanate; 4-(methylsulfinyl)butyl isothiocyanate (sulforaphane); 10-(methylsulfinyl)decyl isothiocyanate; 7-(methylsulfinyl)heptyl isothiocyanate; 6-(methylsulfinyl)hexyl isothiocyanate; 9-(methylsulfinyl)nonyl isothiocyanate; 8-(methylsulfinyl)octyl isothiocyanate; 7-methylsulfinyl-3-oxoheptyl isothiocyanate; 8-methylsulfinyl-3-oxooctyl isothiocyanate; 5-(methylsulfinyl)pentyl isothiocyanate; 3-(methylsulfinyl)propyl isothiocyanate; 11-(methylsulfinyl)undecyl isothiocyanate; 4-methylsulfonylbut-3-enyl isothiocyanate; 4-(methylsulfonyl)butyl isothiocyanate; 10-(methylsulfonyl)decyl isothiocyanate; 6-(methylsulfonyl)hexyl isothiocyanate; 9-(methylsulfonyl)nonyl isothiocyanate; 8-(methylsulfonyl)octyl isothiocyanate; 5-(methylsulfonyl)pentyl isothiocyanate; 3-(methylsulfonyl)propyl isothiocyanate; 4-methylthiobut-3-enyl isothiocyanate; 4-(methylthio)butyl isothiocyanate; 10-(methylthio)decyl isothiocyanate; 2-(methylthio)ethyl isothiocyanate; 7-(methylthio)heptyl isothiocyanate; 6-(methylthio)hexyl isothiocyanate; 9-(methylthio)nonyl isothiocyanate; 6-methylthio-3-oxohexyl isothiocyanate; 8-(methylthio)octyl isothiocyanate; 8-methylthio-3-oxooctyl isothiocyanate; 5-(methylthio)pentyl isothiocyanate; 3-(methylthio)propyl isothiocyanate; 4-oxoheptyl; 5-oxoheptyl isothiocyanate; 5-oxooctyl isothiocyanate; 4-oxopentyl or 3-(methylcarbonyl)propyl isothiocyanate; pent-1-enyl isothiocyanate; pent-4-enyl isothiocyanate; n-pentyl isothiocyanate; phenyl isothiocyanate; 4-phenylbutyl isothiocyanate; 2-phenylethyl isothiocyanate; 3-phenylpropyl isothiocyanate; 2-propenyl (allyl isothiocyanate); 2-(α-L-rhamnopyranosyloxy)benzyl) isothiocyanate; 4-(α-L-rhamnopyranosyloxy)benzyl) isothiocyanate; 6-sinapoyl-β-D-1-thioglucoside of 4-methylsulfinylbut-3-enyl isothiocyanate; 4,5,6,7-tetrahydroxydecyl isothiocyanate; 3,4,5-trimethoxybenzyl isothiocyanate; or a derivative thereof.

The oil-based isothiocyanate composition may be used as a nutritional or dietary supplement, a nutraceutical, or as a component of a pharmaceutical composition. The pharmaceutical composition comprising the oil produced by the above method may further comprise a pharmaceutically acceptable excipient, wherein said pharmaceutical composition is selected from the group consisting of, but not limited to, antibiotic, antifungal, antihistamine, anti hypertensive, anti-protozoal, antifilarial, anti-malarial, anti-schistosomal, anti-ulcer, anti-coagulant, anti-anxiety, anti-inflammatory, antiseptic, nematocidal, antiviral, vasodilators, and protective/prophylactic, and can be administered orally, nasally, parenterally, intrasystemically, intraperitoneally, topically (as by drops of transdermal patch), bucally, or as an oral or nasal spray, and wherein said pharmaceutical composition can be used for human or veterinary applications.

A food or drink product, supplement or additive comprising the oil produced by the above method may include, but is not limited to, the group consisting of juices, smoothies, shakes, teas, soups, sauces, salads, granolas, cereals, breads, other baked goods, fried goods, pills, sprays and other ingestible products, and wherein said food or drink product, supplement or additive can be used for human or veterinary applications.

Also provided herein is a skin or hair product comprising the oil produced by the above method. Said skin or hair product may be selected from the group consisting of hair detergents such as shampoo, rinse, conditioning shampoo, and the like; various hair cosmetics including hair lotion, hair conditioner, hair treatment, hair cream, hair spray, hair liquid, hair wax, hair water, hair-styling preparation, perming liquid, hair color, acidic hair color, hair manicure, etc.; or various skin cosmetics such as skin lotion, milky lotion, face wash, makeup remover, cleansing lotion, emollient lotion, nourishing cream, emollient cream, massage cream, cleansing cream, body shampoo, hand soap, bar soap, shaving cream, sunburn cosmetics, deodorant gel, deodorant powder, deodorant lotion, deodorant spray, anti-perspirant gel, anti-perspirant powder, anti-perspirant lotion, anti-perspirant spray, combination deodorant and anti-perspirant gel, combination deodorant/anti-perspirant powder, combination deodorant/anti-perspirant lotion, combination deodorant/anti-perspirant spray, makeup removing gel, moisture gel, moisture essence, UV-preventing essence, shaving foam, face powder, foundation, lipstick, cheek rouge, eyeliner, eye shadow, eyebrow pencil, bathing preparation, etc.; mouth detergent such as toothpaste; anti-aging, wrinkle reduction, resolution of fine lines, skin smoothing, treatment of dark circles, antioxidant protection, skin brightening, discoloration treatment, acne treatment, skin whitening, blemish control, anti-redness, sunscreen, UV protection, anti-redness, moisturizing, hydration, damage repair, pollution defense, etc. These could be creams, gels, serums, masks, face washes, cleansers, exfoliators, scrubs, toners, peels, mists, wipes, etc., and other hair and skin products, and wherein said skin or hair product can be used for human or veterinary applications.

Also provided herein is a method of inducing the Nrf2/Keap1 Phase 2 response in a mammal comprising administering an effective amount of the above pharmaceutical composition to a mammal in need thereof. The “Phase 2 response” is defined herein as the suite of cryoprotective, detoxification and antioxidant proteins, peptides, and enzymes elicited in a mammal following administration of isothiocyanates.

Also provided herein is a method of inducing the Nrf2/Keap1 Phase 2 response in a mammal comprising administering an effective amount of the above food or drink product, dietary supplement, nutraceutical or additive to a mammal in need thereof.

Also provided herein is a method of inducing the Nrf2/Keap1 Phase 2 response in a mammal comprising administering an effective amount of the above skin or hair product to a mammal in need thereof.

Also provided herein is a method of inducing the Nrf2/Keap1 Phase 2 response in a plant comprising administering an effective amount of the agricultural product described herein to a plant or to soil or plant growth substrate in need thereof.

Also provided herein is a method for treating or preventing skin cancer in a mammal comprising administering the above skin product to the skin of said mammal.

Also provided herein is a method of treating or preventing allergic, neurodevelopmental, neurodegenerative or inflammatory responses, leading to arterial occlusion, Alzheimer's Disease, Parkinson's Disease, Autism, Huntington's Disease, schizophrenia, amyotrophic lateral sclerosis (ALS), cancer, diabetes, metabolic syndrome, liver disease, hepatitis, hypocholesterolemia, chronic gastritis, hypertension, joint inflammation (arthritis), macular degeneration, peptic and duodenal ulcers and gastritis, stroke, injury-related dysfunction including traumatic brain injury & spinal cord injury, ischemia-reperfusion induced brain injury, hemorrhagic stroke, chronic obstructive pulmonary disease (COPD), emphysema, asthma, smoking-related cardiomyopathy, uveitis, and upper airway disease in a mammal comprising administering an effective amount of the pharmaceutical composition described herein to said mammal.

### BRIEF DESCRIPTION OF THE DRAWINGS

FIG. 1 is a process diagram illustrating an exemplary process for producing an isothiocyanate-containing oil product as described herein.

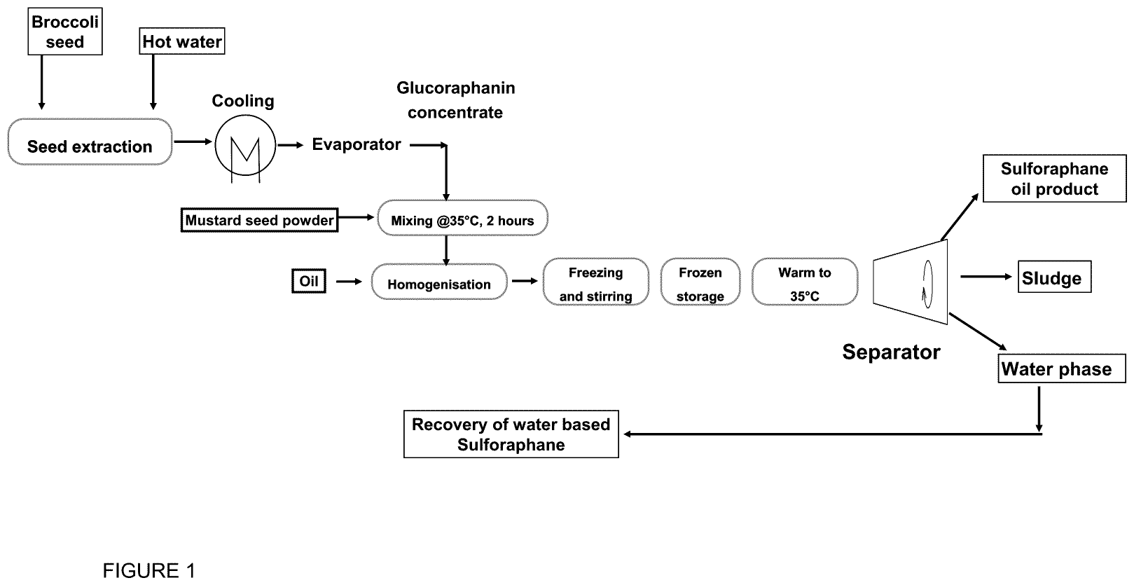

### DETAILED DESCRIPTION

### Definitions

In the description and tables which follow, several terms are used. To provide a clear and consistent understanding of the methods and compositions described herein, the following definitions are provided.

A chemoprotector or chemoprotectant is a synthetic or naturally occurring chemical agent that reduces susceptibility in a mammal to the toxic and neoplastic effects of carcinogens.

A phytochemical is a naturally occurring, non-nutritive plant chemical that has protective or disease preventive properties to mammals. Phytochemicals perform some of the following biochemical processes (but not limited to these): antioxidant activity, detoxification, anti-inflammatory activity, hormonal, stimulation of different enzymes, interference with DNA replication, and antibacterial or antifungal effects.

A monofunctional inducer increases the activity of the Nrf2/Keap 1 Phase 2 enzymes selectively without significantly altering Phase 1 enzyme activities. Monofunctional inducers do not depend on a functional Ah receptor but enhance transcription of Phase 2 enzymes by means of an Antioxidant Response Element (ARE) also known as the Electrophile Response Element (EpRE). Sulforaphane is a monofunctional inducer.

A bifunctional inducer increases 1) activities in both Phase 1 enzymes, such as cytochromes P-450, and Phase 2 enzymes, and 2) requires the participation of an Aryl hydrocarbon (Ah) receptor and its cognate Xenobiotic Response Element (XRE). Examples include flat planar aromatics such as polycyclic hydrocarbons, azo dyes, or 2,3,7,8-tetrachloro-dibenzo-p-dioxin (TCDD).

Glucosinolates, which are well known in the art, and are phytochemicals which occur in all plant tissues of 16 separate higher plant families, and convert to bioactive isothiocyanates via enzymatic hydrolysis. Glucosinolates are grouped as either aliphatic, aromatic, or indole forms. Enzymatic hydrolysis of glucosinolates yields nitriles, epithionitriles, thiocyanates, and/or isothiocyanates depending on the parent glucosinolate, pH and other factors.

Examples of glucosinolates include, but are not limited to 1-acetyl-indol-3-methyl glucosinolate; 4-(4′-O-acetyl-α-L-rhamnosyloxy)benzyl glucosinolate; 3-O-apiosylglucomatronalin glucosinolate; 3-O-apiosylglucomatronalin 3,4-dimethosybenzoyl ester glucosinolate; 2-(α-L-arabinopyranosyloxy)-2-phenylethyl glucosinolate; 4-(benzoyloxy)butyl glucosinolate; 2-(benzoyloxy)ethyl glucosinolate; 6-(benzoyloxy)hexyl glucosinolate; 5-(benzoyloxy)pentyl glucosinolate; 2-benzolyloxy-1-ethylethyl glucosinolate; benzoyloxymethyl glucosinolate; 2-benzoyloxy-1-methylethyl glucosinolate; 3-benzoyloxypropyl glucosinolate; benzyl glucosinolate; but-3-enyl glucosinolate; n-butyl glucosinolate; 3,4-dihydroxybenzyl glucosinolate; 3,4-dimethoxybenzyl glucosinolate; 2,2-dimethyl-2-(4-methoxyphenyl)ethyl or 2-(4-methoxyphenyl)-2,2-dimethylethyl glucosinolate; ethyl glucosinolate; 1-ethyl-2-hydroxyethyl glucosinolate; 4-(α-L-glucopyranosyloxy)benzyl) glucosinolate; 6-heptenyl glucosinolate; 5-hexenyl glucosinolate; n-hexyl glucosinolate; 2-hydroxybenzyl glucosinolate; 3-hydroxybenzyl glucosinolate; 4-hydroxybenzyl glucosinolate; 2(R)-hydroxybut-3-enyl glucosinolate; 2(S)-hydroxybut-3-enyl glucosinolate; 3-hydroxybutyl glucosinolate; 4-hydroxybutyl glucosinolate; 2-hydroxyethyl glucosinolate; 4-hydroxyindol-3-ylmethyl glucosinolate; 2-hydroxy-2-methylbutyl glucosinolate; 1-(hydroxymethyl)propyl glucosinolate; 2-hydroxy-2-methylpropyl glucosinolate; 3-hydroxy-6-(methylsulfinyl)hexyl glucosinolate; 3-hydroxy-5-(methylsulfinyl)pentyl glucosinolate; 3-hydroxy-6-(methylsulfonyl) hexyl glucosinolate; 3-hydroxy-5-(methylsulfonyl)pentyl glucosinolate; 3-hydroxy-6-(methylthio)hexyl glucosinolate; 3-hydroxy-5-(methylthio)pentyl glucosinolate; 2-hydroxypent-4-enyl glucosinolate; 2-hydroxypentyl glucosinolate; 2(R)-hydroxy-2-phenylethyl glucosinolate; 2-hydroxypropyl glucosinolate; 3-hydroxypropyl glucosinolate; indol-3-ylmethyl glucosinolate; “iso”-heptyl glucosinolate; “iso”-hexyl glucosinolate; 2-methoxybenzyl glucosinolate; 3-methoxybenzyl glucosinolate; 4-methoxybenzyl glucosinolate; 1-methoxyindol-3-ylmethyl glucosinolate; 4-methoxyindol-3-ylmethyl glucosinolate; 2-(4-methoxyphenyl)-2-hydroxyethyl glucosinolate; methyl glucosinolate; 3-methyl-3-butenyl glucosinolate; 1-methylbutyl glucosinolate; 2-methylbutyl glucosinolate; 3-methylbutyl glucosinolate; 3-methylcarbonylpropyl glucosinolate; 1-methylethyl (isopropyl) glucosinolate; 1-methyl-2-hydroxyethyl glucosinolate; 3-methylpentyl glucosinolate; 4-methylpentyl glucosinolate; 2-(4-methoxyphenyl)-2-hydroxyethyl glucosinolate; 2-methyl-2-propenyl glucosinolate; 1-methylpropyl (sec-butyl, 2-butyl) glucosinolate; 2-methylpropyl (isobutyl, 2-propyl) glucosinolate; 4-methylsulfinylbut-3-enyl glucosinolate; 4-(methylsulfinyl) butyl glucosinolate (sulforaphane); 10-(methylsulfinyl)decyl glucosinolate; 7-(methylsulfinyl) heptyl glucosinolate; 6-(methylsulfinyl)hexyl glucosinolate; 9-(methylsulfinyl)nonyl glucosinolate; 8-(methylsulfinyl)octyl glucosinolate; 7-methylsulfinyl-3-oxoheptyl glucosinolate; 8-methylsulfinyl-3-oxooctyl glucosinolate; 5-(methylsulfinyl)pentyl glucosinolate; 3-(methylsulfinyl)propyl glucosinolate; 11-(methylsulfinyl)undecyl glucosinolate; 4-methylsulfonylbut-3-enyl glucosinolate; 4-(methylsulfonyl)butyl glucosinolate; 10-(methylsulfonyl)decyl glucosinolate; 6-(methylsulfonyl)hexyl glucosinolate; 9-(methylsulfonyl)nonyl glucosinolate; 8-(methylsulfonyl)octyl glucosinolate; 5-(methylsulfonyl)pentyl glucosinolate; 3-(methylsulfonyl)propyl glucosinolate; 4-methylthiobut-3-enyl glucosinolate; 4-(methylthio)butyl glucosinolate; 10-(methylthio)decyl glucosinolate; 2-(methylthio)ethyl glucosinolate; 7-(methylthio)heptyl glucosinolate; 6-(methylthio)hexyl glucosinolate; 9-(methylthio)nonyl glucosinolate; 6-methylthio-3-oxohexyl glucosinolate; 8-(methylthio)octyl glucosinolate; 8-methylthio-3-oxooctyl glucosinolate; 5-(methylthio)pentyl glucosinolate; 3-(methylthio)propyl glucosinolate; 4-oxoheptyl; 5-oxoheptyl glucosinolate; 5-oxooctyl glucosinolate; 4-oxopentyl or 3-(methylcarbonyl)propyl glucosinolate; pent-1-enyl glucosinolate; pent-4-enyl glucosinolate; n-pentyl glucosinolate; phenyl glucosinolate; 4-phenylbutyl glucosinolate; 2-phenylethyl glucosinolate; 3-phenylpropyl glucosinolate; 2-propenyl (allyl glucosinolate); 2-(α-L-rhamnopyranosyloxy)benzyl) glucosinolate; 4-(α-L-rhamnopyranosyloxy)benzyl) glucosinolate; 6-sinapoyl-β-D-1-thioglucoside of 4-methylsulfinylbut-3-enyl glucosinolate; 4,5,6,7-tetrahydroxydecyl glucosinolate; and 3,4,5-trimethoxybenzyl glucosinolate.

Isothiocyanates are released through enzymatic hydrolysis of glucosinolates by myrosinase. Isothiocyanates are compounds containing the thiocyanate (SCN) moiety and are easily identifiable by one of ordinary skill in the art. The description and preparation of isothiocyanate analogs is described in United States Reissue Patent 36,784, and is hereby incorporated by reference in its entirety. An example of an isothiocyanate includes, but is not limited to, sulforaphane (4-methylsulfinylbutyl isothiocyanate or (−)-1-isothiocyanato-4(R)-(methylsulfinyl) butane) or its analogs. Isothiocyanates may be produced from glucosinolates via the action of the enzyme myrosinase. Myrosinase is found in the same plants that are sources of glucosinolates, for example, the mustards (including white mustard, Sinapis alba, Chinese mustard, Brassica japonica; black mustard, Sinapis nigra or Brassica nigra; field mustard, Brassica campestris; Ethiopian or Abyssinian mustard, Brassica carinata; and brown mustards), horseradish, arugula (or rocket or roquette, Eruca spp.), cress, peppergrass (Lepidium virginicum), pennycress (Thlaspi arvense), garden cress (Lepidium sativum), mignonette (Reseda odorata), wasabi (Wasabia japonica), daikon (Raphanus sativus), dyer's rocket (R. luteola), moringa (Moringa oleifera), cabbage tree (M. stenopetala), Arabidopsis spp., Capsella spp., Cardaria spp., yellow mustard (Brassica juncea), rape seed (Brassica napus), common dietary brassicas like broccoli, Brussels sprouts, cauliflower, cabbage, bok choy, kale, charlock, Chinese cabbage, Chinese kale, collards, colza, kohlrabi, mizuna, swede or rutabaga, tendergreen, texsel greens, tronchuda cabbage, turnip, turnip rape, and recently developed hybrid vegetables such as Broccolini®, Asparation®, and Broccoflower.

An epithiospecifier protein (ESP) is a protein that catalyzes formation of nitriles or epithionitriles during glucosinolate hydrolysis by myrosinase. After myrosinase hydrolysis, epithionitriles can be generated by the ESP protein in the presence of iron and a favorable pH; however, in the absence of ESP, glucosinolates convert to isothiocyanates. Heating of a plant material, such as broccoli, for 10 minutes at 140° F., irreversibly inactivates the ESP protein while not affecting the enzymatic activity of myrosinase, in turn, maximizing the conversion of glucosinolate to its cognate isothiocyanate.

An electrophile is a molecule that has a positively charged center, so that it can react with electron-rich centers such as those that exist in DNA and cause damage. Many cancer-causing chemicals are electrophiles or converted to electrophiles.

Glutathione (GSH) is a naturally occurring peptide, serving as a biological redox agent or a coenzyme, present in very high concentrations in cells. It is the principal endogenous natural antioxidant that protects cells against oxidative damage.

A glucosinolate concentration is the average amount of glucosinolate produced per gram of selected plant material.

Inducer activity or Phase 2 enzyme-inducing activity is a measure of the ability of a compound(s) to induce Phase 2 enzyme activity.

Inducer potential or Phase 2 enzyme-inducing potential is a measure of the combined amounts of inducer activity in plant tissue provided by isothiocyanates, plus glucosinolates that can be converted by myrosinase to isothiocyanates. Glucosinolates are not themselves direct inducers of mammalian Phase 2 enzymes; instead, their metabolic products, isothiocyanates, are inducers. The inducer potential, as distinct from inducer activity, of plant extracts can be measured by adding purified myrosinase, obtained from the same, or other plant sources, to an assay system. Inducer potential can be measured using a multiwell plate screen with murine hepatoma cells for in vitro measurement of NQO1 specific activity.

Plant material is defined as plant tissue, whole plants, and plant parts consisting of seeds, fruit, sprouts, leaves, stems, tubers, flowers and roots.

A mammal is defined as an endothermic or “warm-blooded” vertebrate animal that has the presence of 1) mammary glands, which in the females produces milk for the nourishment of the young, and 2) hair or fur. The mammal class includes about 5500 species, 1200 genera, 152 families and 46 orders. The class of mammals includes human beings, apes, many four-legged animals, whales, dolphins, and bats.

Non-aqueous or organic solvents are chemicals in which other organic compounds readily dissolve; compounds which are also miscible in water are commonly used to improve the solubility of organic compounds in aqueous solutions. In the context of the present application, the terms “nonaqueous solvent” and “organic solvent” are used interchangeably and refer to the same substances. Commonly used non-aqueous/organic solvents include, but are not limited to, alcohols (e.g., ethanol, isopropanol, t-butyl alcohol, lauryl alcohol, benzyl alcohol, cyclohexanol), alkanes (e.g., hexane, heptane), lauryl lactate, benzyl benzoate, ethyl hexyl lactate, tetraglycol, N-1-methyl-2pyrrolidone, dimethyl sulfoxide, polyethylene glycol, glycerin, and glycerol.

A composition which is substantially free of non-aqueous solvents will comprise no non-aqueous solvent other than that incidentally present in the other ingredients in the composition; for example, less than 1% by weight of a non-aqueous solvent, for example less than 0.9%, less than 0.8%, less than 0.7%, less than 0.6%, less than 0.5%, less than 0.4%, less than 0.3%, less than 0.2%, or less than 0.1% by weight of non-aqueous solvent.

A leachate is defined as a liquid containing soluble material which was removed from a solid mixture through which liquid was passed.

Hydrolysis is a chemical reaction in which a compound reacts with water, causing decomposition and results in the production of two or more compounds.

Enzymatic hydrolysis is a chemical reaction in which a compound reacts with water and an enzyme, causing decomposition and results in the production of two or more compounds. A glucosinolate may be converted to its cognate isothiocyanate by the enzyme, myrosinase.

A vehicle, in the pharmaceutical industry, is defined as an inactive substance blended with a drug or active substance which enables for easier application, ingestion of administering of the active substance.

Unless otherwise specified, reference to plants and/or plant parts herein will be understood to refer to the entire plant, and/or to any part of the plant, including roots, stems, leaves, seeds, bark, flowers, and flower parts.

Emulsifiers, solubilizers, and surfactants enable oil and water to be combined to form products such as creams, lotions, and the like. Emulsifiers, solubilizers, and surfactants may include, but are not limited to, brassica alcohols, cetostearyl alcohol, cetyl alcohol, cocamidopropyl betaine, coco glucoside, decyl glucoside, glycerol monostearate, Lamesoft® PO 65, lauryl glycoside, polyglycerol 4 oleate, polysorbate 20, sodium coco sulfate, polysorbate 80, sodium cocoyl isethionate, soya lecithin, sodium lauryl sulfoacetate (SLSA), stearic acid, and lecithin.

Organic solvents and solubilizing agents do not encompass traces of diatomaceous earth, anhydrous salts, or various other fining agents and filter aids known and used in the art to dewater and clarify oil.

A pharmaceutical product is any preparation containing an isothiocyanate extracted into oil from a glucosinolate-containing plant which can deliver that isothiocyanate to the mammal administered the pharmaceutical product. The pharmaceutical product can be an antibiotic, antifungal, antihistamine, anti hypertension, anti-protozoal, antifilarial, anti-malarial, anti-schistosomal, anti-ulcer, anti-coagulant, anti-anxiety, anti-inflammation, antiseptic, vasodilator, protective/prophylactic, or other pharmaceutical product. The pharmaceutical product can be administered orally, nasally, parenterally, intrasystemically, intraperitoneally, topically (as by drops of transdermal patch), bucally, sublingually, or as an oral or nasal spray. The term “parenterally” refers to the modes of administration which include intravenous, intramuscular, intraperitoneal, intracisternal, subcutaneous and intraarticular injection and infusion. Solid dosage forms of the pharmaceutical product include, but are not limited to, capsules, dragees, tablets, pills, powders and granules. Liquid dosage forms of the pharmaceutical product include, but are not limited to pharmaceutically acceptable emulsions, solutions, suspensions, syrups and elixirs. As described herein, the pharmaceutical product can be used for either human or veterinary applications.

A food or drink product, dietary supplement, nutraceutical or additive is any ingestible preparation containing an isothiocyanate extracted into oil from a glucosinolate-containing plant which is capable of delivering that isothiocyanate to the mammal ingesting the food or drink product, dietary supplement, nutraceutical or additive, from the group consisting of, but not limited to, juices, smoothies, shakes, teas, soups, sauces, salads, granolas, cereals, breads, other baked goods, fried goods, capsules, tablets, pills, sprays or other ingestible products, dietary supplements, nutraceuticals or additives. The dose of isothiocyanate added to a food or drink product, supplement or additive preferably is in the range of 1 μmol to 1,000 μmol per serving. However, the dose of glucosinolate and/or isothiocyanate supplementing the food product can be higher. As described herein, the food or drink product, dietary supplement, nutraceutical or additive can be used for either human or veterinary applications.

A skin or hair product is any dermal preparation containing an isothiocyanate extracted into oil from a glucosinolate-containing plant which is capable of delivering those isothiocyanates to the mammal being administered the skin or hair product. The skin or hair product can be, but is not limited to, hair detergents such as shampoo, rinse, rinse-in-shampoo, conditioning shampoo, and the like; various hair cosmetics including hair lotion, hair conditioner, hair treatment, hair cream, hair spray, hair liquid, hair wax, hair water, hair-styling preparation, perming liquid, hair color, acidic hair color, hair manicure, etc.; or various skin cosmetics such as skin lotion, milky lotion, face wash, makeup remover, cleansing lotion, emollient lotion, nourishing cream, emollient cream, massage cream, cleansing cream, body shampoo, hand soap, bar soap, shaving cream, sunburn cosmetics, deodorant gel, deodorant powder, deodorant lotion, deodorant spray, anti-perspirant gel, anti-perspirant powder, anti-perspirant lotion, anti perspirant spray, combination deodorant & anti-perspirant gel, combination deodorant/anti-perspirant powder, combination deodorant/anti-perspirant lotion, combination deodorant/anti-perspirant spray, makeup removing gel, moisture gel, moisture essence, UV-preventing essence, shaving foam, face powder, foundation, lipstick, cheek rouge, eyeliner, eye shadow, eyebrow pencil, bathing preparation, etc.; mouth detergent such as toothpaste and mouth wash; other skin products such as anti-aging, wrinkle reduction, resolution of fine lines, skin smoothing, treatment of dark circles, antioxidant protection, skin brightening, discoloration treatment, acne treatment, skin whitening, blemish control, anti-redness, sunscreen, UV protection, anti-redness, moisturizing, hydration, damage repair, pollution defense, etc. These could be creams, gels, serums, masks, face washes, cleansers, exfoliators, scrubs, toners, peels, mists, wipes, etc.; or other skin and hair products. In these embodiments, the skin or hair product can be used for either human or veterinary applications.

An agricultural product is any preparation containing an isothiocyanate extracted into oil from a glucosinolate-containing plant which is capable of delivering those isothiocyanates to the agricultural system being treated. The agricultural product can be agricultural pesticides, powders, pellets, sprays, fertilizers, composts, soil amendments, in-furrow applications, or other agricultural products.

Some examples of edible oils (also referred to as digestible oils) for use in the processes and compositions described herein include: vegetable, fruit, nut, or seed oils (such as cacao butter, coconut oil, peanut oil, soybean oil, safflower seed oil, corn oil, olive oil, castor oil, cottonseed oil, arachis oil, chia seed oil, sunflower seed oil, fractionated coconut oil, palm oil, pomegranate seed oil, rapeseed (canola) oil, grape seed oil, wheat germ oil, sesame oil, avocado oil, algal oils, sweet almond oil, flax seed oil, mustard seed oil, borage oil, macadamia nut oil, orange oil (D-Limonene), peppermint oil, perilla seed oil, Sea Buckthorn fruit oil and Sacha Inchi oil, and animal oils (such as fish oil, cod liver oil, lanolin oil, Cera Alba (beeswax) and emu oil).

Some examples of topical oils (oils unsuitable for oral consumption, but important for skin care, personal care and cosmetic applications) for use in the processes and compositions described herein include: fruit, nut, leaf, essential, root or seed oils (such as plum and apricot kernel oils, argan oil, baobab oil, evening primrose oil, jojoba oil, lemongrass essential oil, mango seed oil, marula fruit oil, Meadowfoam oil, Mongongo Oil, Moringa oil, shea nut oil, rosehip seed oil, Tamanu oil, etc.).

### Composition Comprising Glucosinolates in Oil

Provided herein is a composition comprising an isothiocyanate and edible or topical oil, wherein the composition is substantially free of water, solubilizing agents, stabilizers, and organic solvents. “Substantially free of water” means that the composition comprises an amount of water by weight % of the composition less than 5%, less than 4%, less than 3%, less than 2%, or less than 1%. “Substantially free of solubilizing agents and organic solvents” means that the composition comprises an amount of one or more solubilizing agents or organic solvents by weight % of the composition which is less than 1%, less than 0.5%, less than 0.1%, less than 0.05%, or less than 0.01%. In one embodiment the composition will have no detectable solubilizing agent or organic solvent. The person of ordinary skill in the art will recognize that, because of the ubiquity of water in the environment, and in the plant materials from which isothiocyanates are typically isolated, it can be difficult to prepare a product which has no measurable water. The presence of detectable amounts of solubilizing agents and organic solvents can easily be avoided by preparing the composition without using such solubilizing agents and organic solvents.

The concentration of isothiocyanate in the composition described may be expressed as a percent of isothiocyanate by weight. In one embodiment, the amount of isothiocyanate agent may range from about 0.0001% to about 20% by weight. In another embodiment, the amount may range from about of 1% to about 15% by weight. In yet another embodiment, the amount may range from 3.75% to about 10% by weight. In another embodiment, the amount may range from about 1% to about 99% by weight, from about 10% to about 80% by weight, and more typically, from about 20% to about 60% by weight. In another embodiment, the amount may be about 5% by weight, less than about 5% by weight, less than about 4% by weight, less than about 3% by weight, less than about 2% by weight, or less than about 1% by weight. In another embodiment, the amount may be greater than about 5%, greater than about 10%, greater than about 15%, greater than about 20%, greater than about 25%, greater than about 30%, greater than about 35%, greater than about 40%, greater than about 50%, greater than about 55%, greater than about 60%, greater than about 65%, greater than about 70%, or greater than about 75%. In one embodiment, the composition contains at least about 0.05% (+/−0.002%) isothiocyanate by weight of the composition.

Any isothiocyanates may be used in the compositions described herein. For example, isothiocyanates for use in the composition described herein may include, but are not limited to, allyl isothiocyanate, sulforaphane and its metabolites, benzyl isothiocyanate, phenylethyl isothiocyanate, and mixtures thereof. In an exemplary embodiment, the isothiocyanate is sulforaphane.

In one class of embodiments of the compositions described herein, the isothiocyanate(s) are derived from plant sources. Suitable plant sources of isothiocyanates are known to the skilled artisan; most typically, such plant sources include cruciferous vegetables such as the mustards (including white mustard, Sinapis alba, Chinese mustard, Brassica japonica; black mustard, Sinapis nigra or Brassica nigra; field mustard, Brassica campestris; Ethiopian or Abyssinian mustard, Brassica carinata; and brown mustards), horseradish, arugula (or rocket or roquette, Eruca spp.), cress, peppergrass (Lepidium virginicum), pennycress (Thlaspi arvense), garden cress (Lepidium sativum), mignonette (Reseda odorata), wasabi (Wasabia japonica), daikon (Raphanus sativus), dyer's rocket (R. luteola), moringa (Moringa oleifera), cabbage tree (M. stenopetala), Arabidopsis spp., Capsella spp., Cardaria spp., yellow mustard (Brassica juncea), rape seed (Brassica napus), common dietary brassicas like broccoli, Brussels sprouts, cauliflower, cabbage, bok choy, kale, charlock, Chinese cabbage, Chinese kale, collards, colza, kohlrabi, mizuna, swede or rutabaga, tendergreen, texsel greens, tronchuda cabbage, turnip, turnip rape, and recently developed hybrid vegetables such as Broccolini®, Asparation®, and Broccoflower.

Oil for use in the compositions described herein may be any oil known to the art as “edible.” Edible oils will typically be selected from the group consisting of vegetable oils, fruit oils, nut oils, seed oils, animal oils, and mixtures thereof. Examples of suitable vegetable oil, fruit oil, nut oil, or seed oil may be selected from the group consisting of cacao butter, coconut oil, peanut oil, soybean oil, safflower seed oil, corn oil, olive oil, castor oil, cottonseed oil, arachis oil, chia seed oil, sunflower seed oil, fractionated coconut oil, palm oil, pomegranate seed oil, rapeseed (canola) oil, grape seed oil, wheat germ oil, sesame oil, avocado oil, algal oils, sweet almond oil, flax seed oil, mustard seed oil, borage oil, macadamia nut oil, orange oil (D-Limonene), peppermint oil, perilla seed oil, Sea Buckthorn fruit oil and Sacha Inchi oil and mixtures thereof. Examples of suitable animal oil may be selected from the group consisting of fish oil, cod liver oil, lanolin oil, Cera Alba (beeswax), emu oil, and mixtures thereof.

Oil for use in the compositions described herein may be any oil known to the art as “topical.” Topical oils (oils unsuitable for oral consumption, but important for skin care, personal care and cosmetic applications) will typically be selected from the group consisting of fruit, nut, essential, leaf, root or seed oils, such as plum and apricot kernel oils, argan oil, baobab oil, evening primrose oil, jojoba oil, mango seed oil, marula fruit oil, Meadowfoam oil, Mongongo Oil, Moringa oil, lemongrass essential oil, shea nut oil, rosehip seed oil, Tamanu oil, etc.), and mixtures thereof, and inclusive of ‘edible oils’.

Organic Solvent-Free Extraction of Glucosinolates from Plant Material

Provided herein is a process for extracting glucosinolates from plant material which does not require the use of organic solvents. In particular, the process described herein uses water to extract glucosinolates, such as glucoraphanin, from plant material. The process comprises mixing the plant material with hot water to provide an aqueous mixture rich in glucosinolates.

The process described herein may comprise an additional step of preparing the aqueous glucosinolate concentrate by extracting glucosinolate from plant material with hot or boiling water, wherein the water collected is the aqueous glucosinolate concentrate. “Hot” water in this context means water that is at least about 70° C. but is not boiling. Accordingly, “hot” water in this context means water that has a temperature in ° C. of about 70, 71, 72, 73, 74, 75, 76, 77, 78, 79, 80, 81, 82, 83, 84, 85, 86, 86, 88, 89, 90, 91, 92, 93, 94, 95, 96, 97, 98, or 99° C. The step of extracting with hot or boiling water may optionally be carried out more than once. For example, the step of extracting with hot or boiling water may be carried out five times, with each volume of water collected being added to the aqueous glucosinolate concentrate prepared in the previous step(s). Cruciferous plants are known to be high in glucosinolates; accordingly, the plant material for use in the extraction step(s) may advantageously be a cruciferous plant or plant part. Suitable cruciferous plants for use in the process described herein include, but are not limited to, Brassicacae (Cruciferae), Moringaceae and Resedaceae, which collectively include, but are not limited to, broccoli, broccoli sprouts, Brussels sprouts, cabbage, cauliflower, cauliflower sprouts, daikon, horseradish, kale, mustard seed, radish, wasabi, horseradish tree (Moringa oleifera), cabbage tree (M. stenopetala), mignonette (Reseda odorata), dyer's rocket (R. luteola). Other families of plants that contain glucosinolates, which may be employed in the method described herein, include, but are not limited to, Bataceae, Bretschneideraceae, Capparaceae, Caricaceae, Euphorbiaceae, Gyrostemonaceae, Limnanthaceae, Pentadiplandraceae, Phytolaccaceae, Pittosporaceae, Salvadoraceae, Tovariaceae and Tropaeolaceae (and these include plants such as capers (Capparis spinosa), and nasturtium (Tropaeolum majus). For use in the process described herein, plant parts (e.g., seeds) which are high in glucosinolates may be selected. For example, broccoli seeds may be used as the plant material in the process described herein.

Any plant part that contains glucosinolates (e.g. roots, stems, leaves, flowers, seeds, sprouts or seedlings) may be used in this process. Advantageously, plant parts which contain high levels of glucosinolates may be selected for use in this process. Useful plant parts include, but are not limited to, plant seeds. Such plant materials include, but are not limited to, broccoli seeds. At this stage of the process the plant material may be handled in a way that avoids and/or suppresses the activity of any myrosinase which may be present in the plant material. Accordingly, if the plant material is crushed, ground, or flaked prior to mixing with hot water such treatment should be carried out under very dry conditions and at low temperatures (for example, at about 5° C. or less) to suppress myrosinase activity.

Water may be removed from the aqueous mixture to produce a glucosinolate concentrate. For example, water may be evaporated by heating, for example to about 80° C., under reduced pressure. A single step of evaporation may be performed, or more than one evaporation step may be performed to further concentrate the glucosinolate. For example, five rounds of evaporation may be performed. If water is removed by boiling, the water may be boiled for up to about 180 minutes in total; shorter boiling times may also be used, for example, up to about 30 minutes, up to about 45 minutes, up to about 60 minutes, up to about 75 minutes, up to about 90 minutes, up to about 105 minutes, up to about 120 minutes, up to about 135 minutes, up to about 150 minutes, or up to about 165 minutes in total. Alternatively, water may be removed using reverse osmosis.

Next, myrosinase may be added to the glucosinolate concentrate to form a mixture. Myrosinase from any source may be used in the process described herein; in one embodiment, the myrosinase will be from a plant source, which may be refined or, if the myrosinase content is suitably high, may be in the form of the plant material itself. Plants known to have relatively high levels of myrosinase include Sinapis alba, Lepidium sativum (garden cress), Wasabia japonica (wasabi), Raphanus sativus (daikon), Moringa oleifera (moringa), as well as several members of the family Brassicaceae, including Brassica juncea, Brassica napus (rapeseed), and common dietary brassicas like broccoli, cauliflower, cabbage, bok choy, and kale. Papaya seeds are also known to contain myrosinase. In an alternative embodiment, some portion of the myrosinase is extracted from the source plant material prior to combination with the glucosinolate concentrate. Alternatively, synthetic myrosinase may be used. Ascorbic acid (Vitamin C), a cofactor for the myrosinase enzyme, may also be added to the mixture at this point to optimize myrosinase activity.

The mixture may be mechanically mixed by stirring, agitation, or other means, at room temperature or with heating. For example, the mixture may be heated to about 20° C. to about 60° C., for example about 30° C. to about 40° C. In an exemplary embodiment, the mixture is heated to 35° C. and stirred for about two hours. This step results in conversion of the glucosinolates to isothiocyanates. For example, where the glucosinolate is glucoraphanin, exposure to myrosinase will result in the production of sulforaphane.

The isothiocyanate-containing mixture may next be homogenized with an oil to form an emulsion. The oil may be a food-grade vegetable oil or a topical oil. The ratio of oil to the aqueous isothiocyanate-containing mixture may be about 10% oil/about 90% aqueous mixture, about 20% oil/about 80% aqueous mixture, about 30% oil/about 70% aqueous mixture, about 40% oil/about 60% aqueous mixture, about 50% oil/about 50% aqueous mixture, about 60% oil/about 40% aqueous mixture, about 70% oil/about 30% aqueous mixture, about 80% oil/about 20% aqueous mixture, or about 90% oil/about 10% aqueous mixture. Homogenization may be accomplished using any suitable means, for example, by vigorous mixing, or by using a homogenizer such as that typically used for milk.

Following homogenization, the emulsion may be cooled until ice crystals form; for example, to a temperature between about 0° C. and about −40° C.; for example, to a temperature between about −0° C. and about −10° C.; or to a temperature between about −10° C. and about −20° C.; or to a temperature between about −20° C. and about −30° C.; or to a temperature between about −30° C. and about −40° C.; or to about −40° C. The frozen emulsion may then be stored at sub-freezing temperatures (i.e., between about 0° C. and about −40° C.) for an extended period; for example, the emulsion may be stored at sub-freezing temperatures for a period of up to about three weeks, for example, for about one, two, three, four, five, 6, 7, 8, 9, 10, 11, 12, 13, 14, 15, 16, 17, 18, 19, 20, or 21 days.

The emulsion may be then separated into an oil phase, an aqueous phase, and a solid phase. Any suitable means of separating the phases may be employed. For example, If the emulsion has been frozen, water in the form of ice crystals may be removed prior to thawing, by subjecting the frozen mixture to lyophilization. The emulsion may be thawed prior to separation in order to reduce its viscosity. For example, the previously-frozen emulsion may be warmed to room temperature (about 20° C.), or to about 25° C., to about 30° C., to about 35° C., or to about 40° C. Separation of the different phases may be accomplished, for example, using mechanical separating equipment. Osmotically active compounds, such as salts (e.g., NaCl), may be added to the emulsion to alter the osmotic gradient between the phases, thereby changing the relative amounts of isothiocyanate in the three phases. The osmotically active compounds may be “food grade” ingredients. All three phases will contain isothiocyanate.

Following separation, the aqueous phase may be recycled into the mixture for further extraction. This recycling may take place multiple times; for example, the aqueous phase may be recycled into the mixture five times, or more.

Following separation, the solid phase may be frozen and/or lyophilized, in which condition it is suitable for long-term storage at room temperature.

Following the separating step, the aqueous phase may be recycled into the mixture; this recycling step may occur once, or more than once as necessary to harvest as much of the active ingredient as possible; for example, the aqueous phase may be recycled into the mixture five times, up to as many as 20 times. The solid phase may optionally be lyophilized by any suitable method known in the art.

### Dermal Compositions

Isothiocyanates obtained by the process described herein may be employed as active agents in compositions, for example, dermal compositions, which are effective over a wide dosage range. For example, in treating adult humans, compositions and methods of described herein may comprise dosages from 0.01 mg to 1,000 mg, from 0.5 mg to 500 mg, from 1 mg to 100 mg, from 5 mg to 50 mg, and from 10 mg to 25 mg. Alternatively, in treating adult humans, compositions and methods described herein may comprise dosages of lipophilic active agents of 0.01 mg, 0.05 mg, 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg.

In other aspects, the concentration of isothiocyanates within the compositions and methods described herein may range from 5 ppm to about 1000 ppm. In other embodiments, the concentration may range from about 50 to about 500 ppm. In still additional embodiments, the concentration may range from about 50 to about 200 ppm, particularly about 100 ppm.

It is contemplated that the compositions described herein can include any ingredient (e.g., isothiocyanates, carrier, etc.) or any combination thereof described throughout this specification. The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### Example 1

The starting material for this example is 2 kg broccoli seed containing approximately 3-4% glucoraphanin. The broccoli seed is mixed with 6 kg of hot (at least about 70° C.) to boiling water for 30 minutes. This results in 4 kg of water with a glucoraphanin content of 1% (40 g of glucoraphanin, a yield of 50% of the glucoraphanin in the starting plant material).

The glucoraphanin-containing water is then divided into two fractions. For the first fraction, 1 kg of glucoraphanin-containing water from the first step is evaporated in a rotary evaporator at about 80° C. for about 90 minutes, at which time the mass will be reduced to about 140 g. The glucoraphanin content of this first (evaporated) concentrate will be about 7-8% (about 11 g of glucoraphanin).

The second fraction is treated by boiling 2 kg of the glucoraphanin-containing water from the first step at 100° C. for about two hours to reduce the mass to about 375 g. The glucoraphanin content of this second (boiled) concentration is about 4-5% (about 18 g of glucoraphanin).

Next, plant myrosinase and ascorbic acid are added to the evaporated concentrate, and to the boiled concentrate. The MYR/concentrate mixtures are then mechanically mixed for about 90 minutes at about 35° C. The yield of sulforaphane from the evaporated concentrate is about 3-4 g (about 90% recovery), while the yield of sulforaphane from the boiled concentrate is about 6.0 g (about 85% recovery).

Finally, the MYR/concentrate mixtures are mechanically mixed with oil (about 100 g of oil for the evaporated concentrate, about 200 g for the boiled concentrate) for one hour. The amount of sulforaphane in the oil at the end of the hour is about 1 g (about 25% recovery of sulforaphane in oil) for the evaporated concentrate, and about 1.5 g (about 28% recovery) for the boiled concentrate.

### Example 2

The starting material for this example is 200 g of boiled glucoraphanin concentrate (prepared according to Example 1 above) containing approximately 4.75% glucoraphanin (about 9.5 g glucoraphanin). The concentrate is mixed with 10 g mustard seed powder and 100 mg ascorbic acid for about 90 minutes at 35° C. The yield of sulforaphane from the evaporated concentrate is about 3 g (about 90% recovery).

The MYR/concentrate mixtures are mechanically mixed with 200 g oil for one hour. The amount of sulforaphane in the oil at the end of the hour is about 1 g (about 26% recovery).

### Example 3

The starting material for this example is 700 g of wet broccoli seeds containing boiled glucoraphanin concentrate (prepared according to Example 1 above) containing approximately 2% glucoraphanin (about 14 g glucoraphanin), mixed with 100 ml of aqueous glucoraphanin concentrate containing about 7% glucoraphanin (total=about 20 g glucoraphanin). The wet seed/concentrate is mixed with 10 g myrosinase and 100 mg ascorbic acid for about 90 minutes at 35° C., and then mechanically mixed with 300 g oil for one hour. The amount of sulforaphane in the oil at the end of the hour is about 1.8 g (about 24% recovery).

### Example 4

In this experiment glucoraphanin was extracted from plant material into oil using prior art methods without the use of organic solvents or exogenously added solubilizing agents. The broccoli seed used in this experiment is a broccoli seed with a glucoraphanin content of about 4% w/w (approximately 91 micromol/g).

The composition was produced by adding 10 g seed to 90 ml boiling water. The mixture was boiled for 30 minutes, after which the seed was decanted from the water phase, and the water phase cooled to 40° C. A reference sample of the water phase was removed; then 50 mg of mustard seed powder (a known source of myrosinase) was added to 20 ml of the water phase, and incubated for two hours at 40° C. Following incubation, 20 ml of grape seed oil was added to the mixture and shaken to extract SF at 40° C. for about 1 minute. The mixture was allowed to separate into oil and water phases, and samples were taken from each phase. The glucoraphanin (GR) concentration in the reference sample (prior to addition of myrosinase) was 0.525% w/v or 12 mM. At the end of the experiment, the oil phase contained sulforaphane (SF) 0.048% w/v or 2.7 mM and GR 0% or 0 mM; the water phase contained SF 0.143% w/v or 8 mM, and GR 0.030% w/v or 0.675 mM.

The results of this experiment show that an oil-based composition comprising sulforaphane in a concentration of no more than 3 mM can be achieved using prior art methods without the use of organic solvents or exogenously added solubilizing agents.

Although the foregoing refers to particular embodiments, it will be understood that the present invention is not so limited. It will occur to those of ordinary skill in the art that various modifications may be made to the disclosed embodiments and that such modifications are intended to be within the scope of the present invention, which is defined by the following claims. All publications and patent applications mentioned in this specification are indicative of the level of skill of those in the art to which the invention pertains.

All publications and patent applications are herein incorporated by reference to the same extent as if each individual publication or patent application were specifically and individually indicated to be incorporated by reference in its entirety.

What is claimed is:

1. A composition comprising an isothiocyanate and oil, wherein the composition contains at least about 0.05% isothiocyanate by weight of the composition, and wherein the composition is substantially free of water, organic solvents, and exogenously added solubilizing agents.

2. The composition of claim 1, wherein the isothiocyanate is sulforaphane.

3. The composition of claim 1, wherein the oil is an edible oil selected from the group consisting of vegetable oil, fruit oil, nut oil, seed oils, animal oils, and mixtures thereof.

4. The composition of claim 3, wherein the vegetable oil, fruit oil, nut oil, or seed oil is selected from the group consisting of coconut oil, cacao butter, coconut oil, peanut oil, soybean oil, safflower seed oil, corn oil, olive oil, castor oil, cottonseed oil, arachis oil, chia seed oil, sunflower seed oil, fractionated coconut oil, palm oil, pomegranate seed oil, rapeseed (canola) oil, grape seed oil, wheat germ oil, sesame oil, avocado oil, algal oils, sweet almond oil, flax seed oil, mustard seed oil, borage oil, macadamia nut oil, orange oil (D-Limonene), peppermint oil, perilla seed oil, Sea Buckthorn fruit oil and Sacha Inchi oil and mixtures thereof.

5. The composition of claim 3, wherein the animal oil is selected from the group consisting of fish oil, cod liver oil, lanolin oil, Cera Alba (beeswax), emu oil, and mixtures thereof.

6. The composition of claim 3, wherein the oil is a topical oil selected from the group consisting of fruit, nut, essential, leaf, root or seed oils.

7. The composition of claim 6, wherein the topical oil is selected from the group consisting of plum kernel oil, apricot kernel oil, argan oil, baobab oil, evening primrose oil, jojoba oil, mango seed oil, marula fruit oil, Meadowfoam oil, Mongongo Oil, Moringa oil, lemongrass essential oil, shea nut oil, rosehip seed oil, and Tamanu oil, and mixtures thereof.

8. A process for producing a composition according to claim 1, comprising the steps of:
adding hot or boiling water to glucosinolate-containing plant material;
evaporating the water to form a glucosinolate concentrate;
adding (a) plant material containing myrosinase, to the glucosinolate concentrate to form a mixture;
adding oil to the mixture, and homogenizing the mixture to form an emulsion;
freezing the emulsion with stirring;
storing the frozen emulsion at a temperature below about 0° C.;
warming the frozen emulsion to about 30-about 40° C.; and
separating the emulsion in a separator into an aqueous phase, an oil phase, and a solid phase.

9. The process of claim 8 wherein the glucosinolate is glucoraphanin.

10. The process of claim 8 wherein the isothiocyanate is sulforaphane.

11. The process of claim 8 wherein the oil is vegetable oil.

12. The process of claim 8 wherein the step of extracting with hot or boiling water is carried out more than once.

13. The process of claim 12, wherein the step of extracting with hot or boiling water is carried out five times.

14. The process of claim 8, wherein the glucosinolate-containing plant material is from a cruciferous plant.

15. The process of claim 14, wherein the glucosinolate-containing plant material is broccoli seed.

16. The process of claim 15, wherein the glucosinolate-containing plant material is from the same species of plant as the myrosinase-containing plant material.

17. The process of claim 16, wherein the glucosinolate-containing plant material and the myrosinase-containing plant material are the same material.

18. The process of claim 8, wherein the myrosinase-containing plant material is from a different species of plant as the glucosinolate-containing plant material.

19. The process of claim 8, wherein the myrosinase is extracted from the myrosinase-containing plant material prior to combination with the glucosinolate concentrate.

20. The process of claim 8, wherein the glucosinolate concentrate is mixed with the oil and myrosinase for about two hours.

21. The process of claim 8, wherein the mixing step takes place at about 250-50° C.

22. The process of claim 8, wherein the mixing step takes place at about 35° C.

23. The process of claim 8, wherein, following the separating step, the aqueous phase is recycled into the mixture at least once.

24. The process of claim 23, wherein the aqueous phase is recycled into the mixture five times.

25. The process of claim 8, wherein, following the separating step, the solid phase is lyophilized.
